(19) United States
(12) Patent Application Publication
(10) Pub. No.: US 2012/0122183 A1
(43) Pub. Date: May 17, 2012

(54) FLOCCULATION WITH DIVALENT SALT AND PHOSPHATE

(75) Inventors: Jakob Rauhe Pedersen, Albertslund (DK); Peter Frode Pind, Herlev (DK)

(73) Assignee: NOVOZYMES A/S, Bagsvaerd (DK)

(21) Appl. No.: 13/382,500

(22) PCT Filed: Jun. 30, 2010
(86) PCT No.: PCT/EP10/59269

§ 371 Date: Jan. 5, 2012

(30) Foreign Application Priority Data
Jul. 9, 2009 (EP) 09165046.5

(51) Int. Cl. C12N 9/20; C12N 9/54; C12N 9/44; C12N 9/52; C12N 9/00; C12N 9/42

(52) U.S. Cl. 435/198; 435/183; 435/209; 435/210; 435/220; 435/221

(57) ABSTRACT

A method of flocculating a bacterial cell, producing a protein of interest, from a fermentation broth, comprising a) diluting the fermentation broth up to 1000% (w/w) with water; b) adding a divalent salt to a concentration in the fermentation broth of more than 10 milli moles per liter diluted fermentation broth; c) adjusting the phosphate concentration to a concentration in the fermentation broth of more than 10 milli moles per liter diluted fermentation broth; d) adjusting the pH of the diluted fermentation broth to a pH within the range of 6.1-10.5; and e) removing the bacterial cells, whereby a protein solution with a turbidity of less than 100 NTU is obtained.

### TECHNICAL FIELD

The present invention relates to a simple and very effective method for flocculation of a bacterial cell, producing a protein of interest, from a fermentation broth.

### BACKGROUND ART

When a protein of a very high purity is needed, e.g., when the protein is to be used within the pharmaceutical area, a very efficient flocculation process is desired. Many known flocculation agents cannot be used because they could end up as impurities in the final pharmaceutical product.

The purpose of this invention is therefore to provide a simple and efficient solution to the above described problem.

### SUMMARY OF THE INVENTION

The inventors have found that it is possible to flocculate a bacterial cell, producing a protein of interest, from a fermentation broth in a very efficient way by using a combination of a divalent salt and a phosphate salt, so we claim:

A method of flocculating a bacterial cell, producing a protein of interest, from a fermentation broth, comprising
a) diluting the fermentation broth up to 1000% (w/w) with water;
b) adding a divalent salt to a concentration in the fermentation broth of more than 10 milli moles per liter diluted fermentation broth;
c) adjusting the phosphate concentration to a concentration in the fermentation broth of more than 10 milli moles per liter diluted fermentation broth;
d) adjusting the pH of the fermentation broth to a pH within the range of 6.1-10.5; and
e) removing the bacterial cells, whereby a protein solution with a turbidity of less than 100 NTU is obtained.

### DETAILED DISCLOSURE OF THE INVENTION

The present invention relates to a simple and very effective method for flocculation of a bacterial cell, producing a protein of interest, from a fermentation broth comprising adding a divalent salt and a phosphate salt, whereafter the bacterial cell is removed and a protein solution with a very low turbidity is obtained.

### Bacterial Cell

The bacterial cell may be a Bacillus cell, e.g., a Bacillus cell selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis; preferably a Bacillus lentus cell, a Bacillus licheniformis cell, or a Bacillus subtilis cell; in particular a Bacillus licheniformis cell.

In another embodiment the bacterial cell may be an E. coli cell, or a Pseudomonas sp. cell, or a Streptomyces sp. cell, in particular a Streptomyces murinus cell or a Streptomyces acidiscabies cell.

### Protein of Interest

According to the present invention the protein of interest may be a peptide or a polypeptide.

A preferred peptide according to this invention contains from 5 to 100 amino acids; preferably from 10 to 80 amino acids; more preferably from 15 to 60 amino acids. A preferred peptide to be recovered according to the invention is brazzein.

A preferred polypeptide may be any protein that may be produced by a bacterial cell. The protein may be insulin, thaxomin, albumin or an enzyme.

In a preferred embodiment the protein of interest is an enzyme to be used for pharmaceuticals.

In a preferred embodiment, the method is applied to a hydrolase (class EC 3 according to Enzyme Nomenclature; Recommendations of the Nomenclature Committee of the International Union of Biochemistry).

In a particular preferred embodiment an enzyme selected from the group consisting of a protease, a lipase, an amylase and a cellulase is preferred:

Proteases: Suitable proteases include those of animal, vegetable or microbial origin. Microbial origin is preferred. Chemically modified or protein engineered mutants are included.

The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Other suitable proteases are the Nocardiopsis proteases described in, e.g., WO 2005/115445 useful for pancreatic enzyme replacement.

Preferred commercially available protease enzymes include Alcalase™, Savinase™, Primase™, Duralase™, Esperase™, and Kannase™ (Novozymes A/S), Maxatase™, Maxacal™, Maxapem™, Properase™, Purafect™, Purafect OxP™, FN2™, and FN3™ (Genencor International Inc.).

Lipases: Suitable lipases include those of bacterial or fungal origin. Chemically modified or protein engineered mutants are included. Examples of useful lipases include Pseudomonas lipases, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), or P. wisconsinensis (WO 96/12012). Other useful lipases may be a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422).

Other suitable lipases are the lipases described in, e.g., WO 2006/136159 useful for pancreatic enzyme replacement.

Amylases: Suitable amylases (alpha and/or beta) include those of bacterial or fungal origin. Chemically modified or protein engineered mutants are included. Amylases include, for example, alpha-amylases obtained from Bacillus, e.g., a special strain of B. licheniformis, described in more detail in GB 1,296,839.

Other suitable amylases are the amylases described in, e.g., WO 2006/136161 useful for pancreatic enzyme replacement.

Commercially available amylases are Duramyl™, Termamyl SC™, Termamyl Ultra™, Stainzyme™, Natalase™ and BAN™ (Novozymes A/S), Rapidase™ and Purastar™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, or Acremonium.

The protein of interest may also be a glucose isomerase such as Sweetzyme™ (Novozymes A/S).

### Fermentation Broth

The present invention may be useful for any fermentation in industrial scale, e.g., for any fermentation having culture media of at least 50 liters, preferably at least 500 liters, more preferably at least 5,000 liters, even more preferably at least 50,000 liters.

The bacterial cell producing the protein of interest may be fermented by any method known in the art. The fermentation medium may be a minimal medium as described in, e.g., WO 98/37179, or the fermentation medium may be a complex medium comprising complex nitrogen and carbon sources, wherein the complex nitrogen source may be partially hydrolyzed as described in WO 2004/003216.

The fermentation may be performed as a batch, a repeated batch, a fed-batch, a repeated fed-batch or a continuous fermentation process.

In a fed-batch process, either none or part of the compounds comprising one or more of the structural and/or catalytic elements is added to the medium before the start of the fermentation and either all or the remaining part, respectively, of the compounds comprising one or more of the structural and/or catalytic elements are fed during the fermentation process. The compounds which are selected for feeding can be fed together or separately to the fermentation process.

In a repeated fed-batch or a continuous fermentation process, the complete start medium is additionally fed during fermentation. The start medium can be fed together with or separately from the structural element feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fermentation broth from a fed-batch fermentation process is preferred.

### Flocculation

The method of the invention may be applied to an untreated fermentation broth or to a fermentation broth that has first been subjected to, but not limited to, e.g., a pH adjustment and/or a temperature adjustment.

According to the present invention the fermentation broth may be diluted up to 1000% (w/w) with water; preferably the fermentation broth may be diluted 10-1000% (w/w) with water; more preferably the fermentation broth may be diluted 100-900% (w/w) with water; more preferably the fermentation broth may be diluted 200-800% (w/w) with water, more preferably the fermentation broth may be diluted 200-800% (w/w) with water, and in particular the fermentation broth may be diluted 300-700% (w/w) with water.

Dilution with water means, according to the present invention, that the dilution medium may be water, or it may be an ultra filtration permeate from the production of the protein of interest, or it may be a recycle of water from the production of the protein of interest, or it may be a condensate from a heater, or it may be any combination of the above mentioned, e.g., a mixture of water and an ultra filtration permeate.

A divalent salt is then added to the fermentation broth, in particular a calcium salt and/or a magnesium salt, e.g., calcium chloride or magnesium chloride. A preferred embodiment is a calcium salt, in particular calcium chloride.

The divalent salt should be added to a concentration in the fermentation broth of more than 10 milli moles per liter; preferably to a concentration in the fermentation broth of 10-100 milli moles per liter; more preferably to a concentration in the fermentation broth of 20-90 milli moles per liter; more preferably to a concentration in the fermentation broth of 30-80 milli moles per liter; more preferably to a concentration in the fermentation broth of 40-70 milli moles per liter; in particular to a concentration in the fermentation broth of 50-70 milli moles per liter.

The dosage of the divalent salt is typically done either in-line, or in a mixing tank, or by any other method known in the art.

The phosphate concentration in the fermentation broth should then be adjusted to a concentration of more than 10 milli moles per liter; preferably to a concentration in the fermentation broth of 10-50 milli moles per liter; more preferably to a concentration in the fermentation broth of 10-40 milli moles per liter; more preferably to a concentration in the fermentation broth of 10-30 milli moles per liter; more preferably to a concentration in the fermentation broth of 10-20 milli moles per liter.

The phosphate concentration is typically adjusted by adding a phosphate salt such as NaH2PO4, Na2HPO4, or H3PO4.

The dosage of the phosphate salt is typically done either in-line, or in a mixing tank, or by any other method known in the art.

The addition of calcium and phosphate causes a pH decrease, normally to a pH below pH 6.0. By shifting the pH to above pH 6, phosphate and calcium precipitate into a solid form with a low solubility. Surprisingly, this precipitate very efficiently entraps the bacterial cells.

Therefore, after the addition of the calcium and the phosphate the pH of the fermentation broth is then adjusted to a pH above 6.0, in particular to a pH between 6.1 and 10.5; preferably to a pH between 6.2 and 10.5; more preferably to a pH between 6.3 and 10.5; more preferably to a pH between 6.4 and 10.5; more preferably to a pH between 6.5 and 10.5; in particular to a pH between 7.0 and 10.0, especially to a pH between 8.0 and 9.0. The pH adjustment may typically be performed by any base known in the art, e.g., NaOH or KOH.

The flocculated bacterial cells are then removed by methods known in the art such as, but not limited to, filtration, e.g., drum filtration, membrane filtration, filter-press dead end filtration, cross-flow filtration, or centrifugation.

After removing the bacterial cells a protein solution with a turbidity of less than 100 NTU (=Nephelometric turbidity units) is obtained; in particular a protein solution with a turbidity less than 90; more preferably a protein solution with a turbidity less than 80; more preferably a protein solution with a turbidity less than 70; more preferably a protein solution with a turbidity less than 60; in particular a protein solution with a turbidity in the range 5-50.

The turbidity is measured as known in the art and according to U.S. Pat. No. 4,198,161, e.g., by a Hach 2100P portable turbidimeter from Hach Lange.

### Subsequent Downstream Operations

The resulting protein solution may then be further processed by methods known in the art. For example, the protein may be recovered by conventional procedures including, but not limited to, further filtration such as ultrafiltration and diafiltration, extraction, spray-drying, evaporation, precipitation or crystallization. The isolated protein may then be further purified and/or modified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction.

The invention is further illustrated in the following examples which are not intended to be in any way limiting to the scope of the invention as claimed.

### Example 1

### Flocculation with Calcium Alone (Bacillus licheniformis/Amylase)

The purpose of this experiment was to demonstrate that the full effect of coagulation cannot be obtained by adding calcium alone (phosphate is required).

Source: The bacillus cell was a Bacillus licheniformis cell containing a Bacillus stearothermophilus amylase (SEQ ID NO: 1 described in WO 2006/136161).

### Fermentation:

We used a 1500 litre scale; used a salt medium containing a protein source employing inorganic nitrogen as the main nitrogen source and glucose dosing as the carbon source.

Because of a residual phosphate concentration from the fermentation, the phosphate concentration was not zero, but 2.8 mM (see Table 1 below).

Calcium was added as CaCl2 (36%). The concentration of calcium was varied between 0-69.5 mM.
pH of the fermentation broth (before adjustment) was 6.7.
pH was, after the addition of the calcium and the phosphate, adjusted to 7.0 by using NaOH.
The flocculated solution was centrifugated for 5 minutes, 3500 rpm (=2493 g).

TABLE 1
Experimental conditions

| Parameter | Setting |
|---|---|
| Dilution (tap water) | 600% |
| PO4 concentration, added | 0 mM |
| PO4 concentration, total | 2.8 mM |
| Ca concentration added | Varied |
| pH adjustment setpoint | pH 7.0 |

TABLE 2
Results

| c(Ca) added | Sludge | Turbidity |
|---|---|---|
| mM | % w/w | NTU |
| 0.0 | 3.6% | 1000 |
| 23.2 | 5.8% | 1000 |
| 46.3 | 9.2% | 631 |
| 69.5 | 9.5% | 360 |

The tap water used for dilution contained in the order of 2-3 mM Ca. This is not accounted for in the experimental data. The maximum for a turbidity reading was 1000 NTU. When stated that the turbidity is 1000 NTU, this therefore effectively means >1000 NTU.
Experiment 1 demonstrates that it is not possible to obtain an enzyme solution with a low turbidity (<100) without the addition of phosphate.

### Example 2

### Flocculation with Calcium and Phosphate (Bacillus licheniformis/Amylase)

The purpose of this experiment was to evaluate the required levels of phosphate to be added, to obtain a suitable coagulation effect. Because the addition of phosphate lowers the pH, the initial pH will vary depending on the amount of phosphate added.

To confirm that the improved coagulation is not just an effect of this difference, two series of samples were prepared: one “as-is” and one where the pH is adjusted to 5.0 after the addition of calcium and phosphate. Both series were then adjusted to the pH set point of this experiment (10.0).

Source: The bacillus cell was a Bacillus licheniformis cell containing a Bacillus stearothermophilus amylase (SEQ ID NO: 1 described in WO 2006/136161).

### Fermentation:

The fermentation and flocculation were performed as described in Example 1 with the following specific conditions:

TABLE 3
Experimental conditions

| Parameter | Setting |
|---|---|
| Dilution (tap water) | 600% |
| PO4 concentration, added | varied |
| PO4 concentration, total | varied |
| Ca concentration added | 55.6 mM |
| pH adjustment setpoint | pH 10.0 (pre-pH = 5.0) |

TABLE 4
With pre-pH adjustment to 5.0.

| c(PO4) totoal | sludge | Turbidity |
|---|---|---|
| mM | % w/w | NTU |
| 2.8 | 9.8% | 1000 |
| 4.0 | 8.1% | 1000 |
| 5.2 | 12.8% | 1000 |
| 7.6 | 11.5% | 191 |
| 11.1 | 13.5% | 73 |
| 14.7 | 13.5% | 16 |
| 20.7 | 13.7% | 47 |

TABLE 5
Without pre-pH adjustment to 5.0.

| c(PO4) total | sludge | Turbidity |
|---|---|---|
| mM | % w/w | NTU |
| 2.8 | 8.3% | 1000 |
| 4.0 | 9.4% | 1000 |
| 5.2 | 10.2% | 1000 |
| 7.6 | 11.2% | 195 |
| 11.1 | 12.6% | 57 |
| 14.7 | 13.3% | 19 |
| 20.7 | 14.4% | 36 |

Example 2 demonstrates a large effect from adding phosphate. It can be seen that a concentration of more than 10 mM of total phosphate was appropriate to obtain a low turbidity (<100 NTU).
It can also be seen from the data in Table 4 and 5 that the pre-pH adjustment does not have a significant impact on the result.

### Example 3

### Flocculation with Calcium and Phosphate at Various pH Values (Bacillus licheniformis/Amylase)

The purpose of Example 3 was to define the pH range in which the coagulation process works.
Source: The bacillus cell was a Bacillus licheniformis cell containing a Bacillus stearothermophilus amylase (SEQ ID NO: 1 described in WO 2006/136161).

### Fermentation:

The fermentation and flocculation were performed as described in Example 1 with the following specific conditions:

TABLE 6
Experimental conditions

| Parameter | Setting |
|---|---|
| Dilution (tap water) | 600% |
| PO4 concentration, added | 13.1 mM |
| PO4 concentration, total | 15.9 mM |
| Ca concentration added | 55.6 mM |
| pH adjustment setpoint | Varied |

TABLE 7
Results

| pH | Sludge | Turbidity |
|---|---|---|
| — | % w/w | NTU |
| 4.51 | 5.4% | 1000 |
| 5.07 | 1.8% | 1000 |
| 5.51 | 3.4% | 1000 |
| 5.97 | 11.5% | 173 |
| 6.50 | 13.5% | 14 |
| 7.06 | 11.6% | 9 |
| 7.53 | 14.1% | 9 |
| 7.98 | 13.3% | 10 |
| 8.53 | 13.7% | 11 |
| 9.01 | 15.2% | 9 |
| 9.47 | 15.7% | 9 |
| 10.01 | 18.9% | 15 |
| 10.59 | 20.9% | 52 |

The experiment demonstrates that the process works very well within pH 6.5-10.5.

### Example 4

### Flocculation with Calcium and Various Phosphate Concentrations and Various pH Values (Bacillus licheniformis/Protease)

The purpose of Example 4 was to show that the invention also works when the protein of interest is a protease.
Source: The bacillus cell was a Bacillus licheniformis cell containing a Norcardiopsis sp protease (SEQ ID NO: 1 described in WO 2005/115445).

### Fermentation:

The fermentation was performed as known in the art, e.g., as disclosed in Example 2 of WO 2001/058276.

### Test of NaH₂PO₄ Concentrations

12% CaCl₂ (34% solution) (w/w) in regard of undiluted fermentation broth was used for all samples.
Samples were prepared with varying NaH₂PO₄ concentrations.
pH was adjusted to 6.5 and samples were centrifuged at 3500 rpm for 3 minutes.
NTU was measured. Results are listed in Table 8.

TABLE 8
Results from using different NaH₂PO₄ concentrations.

| | NaH₂PO₄ | NaH₂PO₄ | | |
|---|---|---|---|---|
| | mM | mM | | |
| | (in culture | (in diluted | pH adjusted | Turbidity |
| Sample | broth | culture broth | — | NTU |
| 1 | 66.7 | 9.2 | 6.55 | 273 |
| 2 | 100.0 | 14.2 | 6.56 | 68 |
| 3 | 133.3 | 19.2 | 6.50 | 41 |
| 4 | 166.7 | 24.2 | 6.51 | 32 |

It can be seen from Table 8 that the NTU improves at increasing NaH₂PO₄ concentrations. No sludge binding was observed.

### Test of Varying pH

A portion of the diluted culture broth was conditioned with the following conditions:
12% CaCl₂ (34% solution) (w/w) and 1.5% NaH₂PO₄ (w/w), both in regard of undiluted fermentation broth.
The pH was gradually increased by adding NaOH, and samples were removed at increasing pH, for centrifugation test. The samples were centrifuged at 3500 rpm for 3 minutes, and NTU was measured. The results are listed in Table 9.

TABLE 9
Results from different pH adjustments.

| | pH | Sludge | Turbidity |
|---|---|---|---|
| Sample | — | % V/V | NTU |
| 1 | 5.72 | 3 | >1000 |
| 2 | 6.01 | 4 | >1000 |
| 3 | 6.33 | 5 | 46.1 |
| 4 | 6.47 | 5 | 37.9 |
| 5 | 6.74 | 5 | 36.3 |
| 6 | 7.05 | 5 | 35.7 |
| 7 | 7.41 | 5 | 34.3 |
| 8 | 7.83 | 5 | 36.2 |

The results from the pH series in Table 9 show that a dramatic effect was encountered around pH 6.33. No sludge binding was observed.

1. A method of flocculating a bacterial cell, producing a protein of interest, from a fermentation broth, comprising
a) diluting the fermentation broth up to 1000% (w/w) with water;
b) adding a divalent salt to a concentration in the fermentation broth of more than 10 milli moles per liter diluted fermentation broth;
c) adjusting the phosphate concentration to a concentration in the fermentation broth of more than 10 milli moles per liter diluted fermentation broth;
d) adjusting the pH of the diluted fermentation broth to a pH within the range of 6.1-10.5; and
e) removing the bacterial cells, whereby a protein solution with a turbidity of less than 100 NTU is obtained.

2. The method according to claim 1, wherein the bacterial cell is a Bacillus cell.

3. The method according to claim 2, wherein the Bacillus cell is a Bacillus licheniformis cell.

4. The method according to claim 1, wherein the bacterial cell is an E. coli cell.

5. The method according to claim 1, wherein the protein of interest is an enzyme.

6. The method according to claim 5, wherein the enzyme is selected from the group consisting of a protease, a lipase, an amylase and a cellulase.

7. The method according to claim 1, wherein the divalent salt is a calcium salt.

8. The method according to claim 1, wherein the divalent salt concentration is in the range of 10-100 milli moles per liter.

9. The method according to claim 1, wherein the phosphate is obtained from NaH2PO4, Na2HPO4, or H3PO4.

10. The method according to claim 1, wherein the phosphate concentration is in the range of 10-50 milli moles per liter.

11. The method according to claim 1, wherein the bacterial cells are removed by filtration or centrifugation.

12. The method according to claim 1, wherein the protein of interest is a pharmaceutical product.

13. The method according to claim 1, wherein the pH is adjusted to a pH within the range 6.5-10.5.
